(19) United States
(12) Patent Application Publication
(10) Pub. No.: US 2005/0234340 A1
(43) Pub. Date: Oct. 20, 2005

(54) Bolus control for contrast imaging with 3D

(76) Inventors: George A. Brock-Fisher, Andover, MA (US); Janice L. Frisa, Groton, MA (US); McKee D. Poland, Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/091,635

(22) Filed: Mar. 28, 2005

Related U.S. Application Data
(60) Provisional application No. 60/558,073, filed on Mar. 31, 2004.

(51) Int. Cl. A61B 8/14

(52) U.S. Cl. 600/458

(57) ABSTRACT

The present invention relates to contrast imaging. More particularly, the present invention relates to apparatus and methods for ultrasound contrast imaging in which a high-MI signal destroys contrast agent in a blood supply region of the heart after previously infused thoroughly with contrast agent enhanced blood, and a portion of the heart other than the blood supply region is imaged with low MI ultrasound to track the decrease in contrast agent containing blood due to perfusion into the “other then the blood supply region” by blood from the blood supply region, as a function of time.

### CROSS REFERENCE TO RELATED CASES

Applicants claim the benefit of Provisional Application Ser. No. 60/558,073, filed Mar. 31, 2004.

The present invention relates generally to ultrasonic diagnostic imaging techniques, and more particularly to ultrasonic diagnostic imaging systems and methods for measuring perfusion using contrast enhancing agents, preferably with 3D scanning.

Diagnostic ultrasound equipment transmits sound energy into the human body and receives the signals that reflect off of tissues and organs, such as the heart, liver, and kidneys. The value of diagnostic ultrasound lies in the wealth of accurate information it can deliver about the heart and other inner organs without risk to the patient. Diagnostic ultrasound provides a window into the body through which medical professionals may see certain manifestations of disease which might other wise go undetected. Conventional ultrasound diagnostic imaging systems emit pulses of sound energy over a plurality of paths and convert returned echoes received from objects on the plurality of paths into electrical signals. The signals generated are sampled to generate ultrasound data from which an ultrasound image can be reconstructed. The process of obtaining the raw data from which the ultrasound data is produced is typically termed “scanning,” “sweeping,” or “steering a beam”.

Real-time ultrasound imaging presents ultrasound images in a rapid sequential format as the scanning is being performed. Scanning is either performed mechanically (by physically oscillating one or more transducer elements) or electronically. While most scanning to date has been mechanical, more sophisticated and more recently developed ultrasound imaging technology typically utilizes electronic in lieu of mechanical scanning. Electronic scanning requires that one or a group of transducer elements be formed as an array which is/are excited by a set of electrical pulses, one pulse per element, timed to construct a sweeping action. And there are numerous imaging modes for diagnostic ultrasound, as known to those skilled in the art. These include without limitation 2D imaging (sometimes referred to as B-mode), M-mode, Doppler, color-flow imaging, and 3D. The various modes help medical professionals measure the size and function of internal organs, for example, in cardiac imaging of the heart and blood vessels (to be discussed in greater detail below).

Ultrasound technology has recently evolved to enable presentation of an image having the appearance of 3D (a three-dimensional object). Those skilled in the art should understand that there are various ways to implement 3D imaging in ultrasound. For example, U.S. Pat. No. 5,993,390, and U.S. Pat. No. 5,191,931, each disclose systems and methods by which a 3D image may be generated using a three-dimensional data matrix of ultrasound image data (from data acquired using spaced planar 2D sweeps), where the desired processing end is an image for display on a two-dimensional surface with an appearance of 3D. Typically, any 3D-like images generated by the bulk of 3D ultrasound imaging systems in use today do not provide 3D in real-time. That is, the 3D image generation available today typically requires that 3D images be generated with the help of “off-line” post-processing. To do this, sequences of regularly spaced planar two-dimensional sweeps are collected as the position of the probe is translated or rotated in some way between scan frames.

Post-processing manipulation of the sequences reconstructs three-dimensional data sets using acquired position information for each two-dimensional scan plane. The resulting three-dimensional data sets are displayed as rendered 3D images, typically on a separate workstation (not in real time), using any of various well-known, computation-intensive rendering techniques. The real-time rendering and display workstation may be integrated with the ultrasound scanner into one system. One such system known to produce real-time 3D images is the Sonos 7500 sold by Philips Medical Systems. Relatively newer 2D transducers are capable of providing sweeps which acquire frames of imaging data defining predetermined 3D shapes, as taught by published U.S. patent application No. 2003/0013955, U.S. Pat. No. 6,443,896, U.S. Pat. No. 6,537,219, U.S. Pat. No. 5,593,390 and U.S. Pat. No. 6,436,048, commonly owned and incorporated by reference herein. Using 3D imaging techniques, a clinician may look simultaneously at several different cut planes within the 3D volume region of tissue to observe anatomical features at different angles and depths. While most prior art 3D imaging require acquisition of data from the entire volume, where newer methods are now known which speed up acquisition in various sub-volumes or regions with the ROI.

Echocardiography is an ultrasound cardiac imaging technique, which progressively evolved from M-mode to the current multifaceted capabilities of 3D echocardiography, Doppler, color-flow Doppler and intravascular imaging. 3D echocardiography evolved from 2D cardiography, which 2D methods use rapid movements of a 1-D ultrasound beam across the heart to provide real-time cross sectional images. That is, in traditional 2D cardiac imaging, the clinician (cardiologist) could observe the motion of a patient's heart, study the opening and closing of valves, measure cardiac size and function, etc. When imaging in M-mode, the system focuses more on producing a trace of an opening or closing of a valve (in cardiac imaging), rather than on an image. M-mode is quite useful in providing for precise quantitative evaluation of cardiac valve and chamber functions.

Doppler facilitates echocardiography by providing information about the velocity and direction of blood flow within the heart and the vascular system. That is, Doppler provides the clinician (e.g., the cardiologist) with an ability to assess the severity of a malfunctioning valve by measuring the blood flow through the valve. For example, a partially obstructed valve produces a high velocity jet of blood, which may be detected and measured with Doppler techniques. Doppler technology forms the basis for color-flow imaging, which superimposes a color representation of blood flow onto a black-and-white (2D) image of the heart and vessels. Color-flow imaging requires the acquisition of Doppler data at different locations (called sample volumes), over time, and over the image plane of an ultrasonic image.

The Doppler data is used (in color-flow imaging) to estimate the phase shift over succeeding transmit events, at each discrete sample volume. The phase shift relates to the velocity of fluid flow in vessels within the body, with the polarity of the shift indicating direction of flow towards and away from the transducer. This information is color coded in accordance with the magnitude of the shift (i.e., its velocity) and its polarity and is then overlaid on a structural image of the image plane. The colors in the image provide an indication of the speed of blood flow and its direction. In a typical coding, blood flowing towards the transducer is red, and way from the transducer is blue.

Color power Doppler focuses on the intensity of received signals, which exhibit a Doppler shift. This type of Doppler-based imaging technology is described, for example, in U.S. Pat. No. 5,471,990 (Thirsk). The Doppler signal intensity is computed for each sample volume in an image plane and is displayed, using a color derived from a color map. Unlike color Doppler velocity imaging, color power Doppler imaging does not exhibit the problems of direction determination, aliasing and low sensitivity (which are characteristic of velocity or color-flow imaging). Color power Doppler simply displays the Doppler signal intensity at a sample volume in a coded color. Hence, both 2D gray scale and 2D color power Doppler displays find use in perfusion studies, that is, situations in which it is desirable to assess blood perfusion in an organ or structure of the body. Doppler, however, has its limitations. For example, while imaging the abdomen, Doppler signals are typically weak. And, for example, for imaging cardiac vasculature, where cardiac motion tends to mask the weaker signal from red blood cells in the flowing blood, Doppler performs inconsistently and does not always provide diagnostic information.

To extend the utility of perfusion imaging, and in particular, cardiac perfusion imaging, researchers, diagnosticians, etc., have developed methods of perfusion imaging using contrast agents, e.g., microbubbles (to be discussed in greater detail below), which does not rely upon Doppler processing to quantify flow, volume, etc. Microbubbles tend to oscillate asymmetrically in alternating high/low pressures of an acoustic wave to generate harmonics (from radial oscillations of the microbubble), which harmonic elements are readily acquired and processes. That is, contrast agents respond non-linearly to ultrasound energy, and techniques are known for optimizing detection of non-linear responses. In particular, this resonant behavior of contrast agent gives rise to a non-linear acoustic response, typically a second harmonic response; but there are also other non-linear responses that can be detected. So relatively new harmonic imaging methods are used to image contrast agent-laden blood, identify volumes of same over time, or in conjunction with fixed time correlated to a bodily function, i.e., ecg, signal. By analyzing said changes in volume over time, blood flow velocity may be determined and communicated to the clinician (flow velocity information).

Microbubble contrast agent characteristics make them useful in contrast imaging both as tracers to show blood flow to or through the imaged tissue, not just quantifying the rate of blood flow to or through the imaged tissue.

A second characteristic of micro-bubbles, which is also useful, is that if excited with a large enough acoustic pressure in the range of contemporary ultrasound systems, the micro-bubbles can be destroyed by disrupting their shells and thereby allowing the encapsulated gas to escape. The gas from the destroyed micro-bubbles then dissolves into the blood stream. J. Powers, et al., ULTRASOUND CONTRAST IMAGING RESEARCH, Medica Mundi, 44/2, pgs. 28-36, November 2000.

Ultrasound cardiac perfusion imaging techniques with contrast agents have developed to take advantage of the primarily linear response behavior of tissue, as distinguished from that of contrast agents, to cancel or attenuate tissue signals (at or around the fundamental), thereby providing an improved image of the contrast agents (at the harmonics of the fundamental) in view of the predominantly harmonic responses generated by the contrast agents. For example, multiple transmit lines are fired along the same line of sight into the body of the patient. The transmit waveform is modified (e.g., in terms of power, phase, or polarity) from line to line to produce a variation in the response received by the ultrasound transducer. These data points are then processed to remove the influence of their linear components, which are primarily related to the tissue signal, to yield data that primarily contains the non-linear response of the contrast agents (e.g., Pulse Inversion Harmonic Imaging, Real Time Perfusion Imaging, Power Pulse Inversion Imaging, etc.).

Regardless of the technique used to image the contrast agent response to ultrasound energy, one of the most significant pieces of information provided by analyzing a non-linear response is the rate of flow (perfusion) of blood (i.e., in myocardial tissue). Because the flow rates are low in myocardial tissue, a general technique has been employed to measure the rate of reperfusion to heart tissue under investigation as follows. For example, if the clinician wanted to view the vasculature of the heart behind the left ventricle, first contrast agent is administered to the patient. After a fixed time, contrast-agent-laden blood will arrive at and enter the right side of the heart, move into the left ventricle and myocardial tissue. The region of interest (ROI) of the myocardium (e.g., behind the LV) containing contrast-agent-laden blood and/or tissue is first imaged with low mechanical index (MI) ultrasound, for example, by a number of planar 2D scans through the ROI. And region is chosen through which contrast-agent-laden blood must flow to feed the ROI.

The system then transmits high intensity (high mechanical Index or MI) ultrasound signals in a destruction plane. The destruction plane is used to scan the region through which the blood must pass to enter the ROI (various techniques for destroying contrast agent are known to those skilled in the art). For example, the destructive scan may be used to destroy the entire contrast agent content within blood present in the left ventricle such that no shadowing effect from unwanted contrast agent (e.g., in the LV) can affect imaging of the myocardium behind the LV. The lack of contrast agent after destruction by such a scan is sometimes referred to as a ‘negative bolus’ effect.

Destructive scanning is stopped (after the contrast agent is destroyed), and the ROI behind the LV is scanned with low MI ultrasound. Because blood is still pumping, fresh contrast-agent-laden blood moves back into the destruction zone (i.e., LV) and reperfuses the myocardium. Typically, prior art cardiac perfusion techniques include monitoring the intensity of the signals returned from the myocardium due to contrast agent, where reperfision is assumed where increasing intensity is detected. This is sometimes known as the ‘destruction-reperfusion technique’. From the reperfusion of contrast agent over time, analysis of concentration vs. time may be made, and estimates of blood flow rate and maximum blood volume in the myocardium may be made, overcoming same limitations of Doppler flow imaging.

For example, U.S. Pat. No. 6,077,225, commonly owned and incorporated by reference herein, teaches a method which utilizes the destruction of contrast agents, and imaging which focuses on reperfusion of blood containing same after destruction (‘destruction-reperfusion technique’), and thereafter, imaging (with low MI) to measure reperfusion into a ROI where the contrast agent has been destroyed. That is, the '225 patent teaches providing contrast-agent-laden blood to a region of interest (ROI), imaging with low MI ultrasound to identify a particular region (destruction region) through which contrast-agent-laden blood must flow perfuse the ROI. The destruction region is then scanned using high MI ultrasound to destroy contrast agent present in blood there. After waiting a predetermined amount of time, corresponding to the time it takes for the blood in the ROI to be filled with the blood in which the contrast agent has been destroyed, i.e., contrast-agent-depleted blood, the ROI is imaged with low MI ultrasound, hopefully avoiding any shadowing caused by imaging through an area perfused with contrast agent.

However, cardiac perfusion imaging using some variation on the destruction-reperfusion technique is not without drawbacks. For example, following the high-energy impulse frames to destroy the contrast agent within the blood in the destruction region, the ventricle is refilled with a high concentration of contrast agent. If the ultrasound beams used to detect the reflow of contrast agent into the myocardium must scan through or proximate the ventricle (behind the left ventricle), and the region of high contrast agent concentration (closer to the transducer), non-linear propagation generated by the contrast agent in the ventricle may distort the transmitted waveforms, the problem the '225 patent intended to address. As discussed in detail above, such distortion can result in a false reading of perfusion (reperfusion) in the myocardium segments distal to the ventricle chamber. Moreover, because most imaging, 2D or 3D, is done by taking sets of 2D planar images, the contrast agent is only destroyed in blood present in the plane of the ultrasound energy at scanning.

Contrast agent in contrast-agent-laden blood, which lies outside of this volume may, due to motion of the heart, ultrasound transducer, etc., be brought into the volume of view and be misinterpreted as perfusion reflow. Generalizing, conventional perfusion imaging techniques using ultrasound contrast agents do not effectively compensate for the effect of the low level presence of micro-bubbles in moving tissue itself. That is, contrast agent within contrast-agent-laden blood lying outside of a volume depleted of contrast agent using destruction-reperfusion may be compelled by simple mechanical motion into the imaging plane. Such unanticipated movement of contrast agent into the image plane can cause degraded images. This degradation can be substantial, particularly where the heart or lungs is being imaged, due to the inherent frequent and rapid motions associated with both organs, and the tendency for contrast agents in solid tissues to move in response to physical movement within the tissue.

Commonly-owned U.S. patent application Ser. No. 10/291,010, incorporated by reference herein, is directed to improving imaging with ultrasound contrast agents, and addresses the problem of contrast-agent-laden blood from surrounding tissue inadvertently moving into a ROI being imaged for perfusion by contrast-agent-laden blood, and affecting the perfusion determination therefore. More particularly, the '010 application discloses an apparatus and method for contrast imaging, by which deleterious effects on imaging by contrast-agent-laden blood from the area surrounding a ROI inadvertently moving into the ROI during scanning are effectively suppressed. The inventive method disclosed therein attempts to image only reperfused contrast agent to increase the accuracy of the imaging of blood flow rates to or through the tissue in the image plane (ROI). Such method, however, like the above-mentioned destruction-reperfusion-based methods, may still be influenced by shadowing occurring due to contrast agent moving into a ROI being imaged.

The present inventions, which can be better understood with reference to the following drawings, attempts to overcome some of the problems inherent in conventional ultrasound contrast imaging techniques. The components in the drawings are not necessarily to scale, emphasis instead being placed upon clearly illustrating the principles of the present invention. The inventions teach perfusion imaging using contrast agents to accurately measure perfusion, including accurately calculating blood flow velocity. For example, the inventions described herein alleviate the undesirable effect of contrast-agent-laden blood, present within tissue surrounding a ROI, from inadvertently moving into a ROI, resulting in a misinterpretation of perfusion. The fundamental difference between prior art techniques and the present invention is that prior art techniques use hi MI destruction in the ROI to destroy agent, and then attempt to image the wash-in of new contrast agent to the ROI. The present invention teaches the destruction of contrast agent flowing into the ROI, and imaging the ROI to detect the washout of contrast agent from the ROI as a consequence of blood flow. And while the '225 patent teaches the use of one transducer for scanning with low MI ultrasound, and one transducer for scanning with high MI ultrasound, the present invention obviates such requirement due to the fact that it is intended to be implemented in 3D with 2D or Matrix arrays.

In one embodiment, the invention includes a process, routine or method for perfusion imaging with ultrasound contrast agents, wherein contrast agent is introduced into a flow of blood feeding a ROI in the heart. After the contrast agent is introduced, the ROI is imaged using low MI ultrasound, and a region (destruction region) is identified through which contrast agent-laden blood must flow to arrive at the ROI. Once the destruction region is identified, the contrast agent present there is destroyed. This creates a negative step function in the concentration of contrast agent that is delivered to the ROI. Immediately thereafter, or simultaneously with the hi MI imaging of the destruction region, the ROI is imaged with low MI ultrasound as blood from the destruction region, depleted of contrast agent-laden blood, flows in. Imaging in this ROI, over time, will detect the washout of contrast agent, which therefore provides for quantifying perfusion in the ROI. And if the ROI is normally behind tissue or for example, the LV, there will be no shadow effect from contrast agent moving into the ROI, or affecting imaging through the destruction region.

The bottom line is that imaging the ROI with low MI ultrasound immediately after the commencement of continuous destruction by high MI scanning in the destruction region provides for observing and quantifying blood velocity by first identifying volumes of contrast-agent-depleted blood, and observing changes to the volumes over time. Because the inventive method provides that low MI scanning may be through tissue normally affected by the presence of contrast agent, it shows a marked improvement over prior art methods because such inadvertently present contrast agent are now prevented from affecting the low MI imaging. Hence, the depletion of contrast agent can be more clearly imaged and accurate estimates of the direction and of the velocity of the contrast agent flow through the bloodstream can be established therefrom.

Moreover, improvements are also set forth herein for readily identifying and automatically tracking a volumetric scan over a biplane. The reference volume may be defined by the depth, and the biplane, and while the scan volume may be readily utilized as the ROI, it may also be utilized as the destruction volume. So, for example, by defining the c-scan as the destruction volume, and arranging its location such that it is located between the transducer and ROI, all contrast agents within the c-scan (or destruction volume) may be destroyed by scanning same destruction volume with high MI frames. The ROI outside of the destruction plane may be imaged using low-MI. The destruction volume should be defined in order to minimize the flow of contrast-agent-laden blood into the ROI, as a result of, for example, organ movement, voluntary or involuntary. That is, we need to destroy contrast agent in blood present in the destruction volume proximate the ROI such that there will be minimal seepage or movement of contrast-agent-laden blood from the tissue into the ROI, which could affect any quantification of perfusion therein.

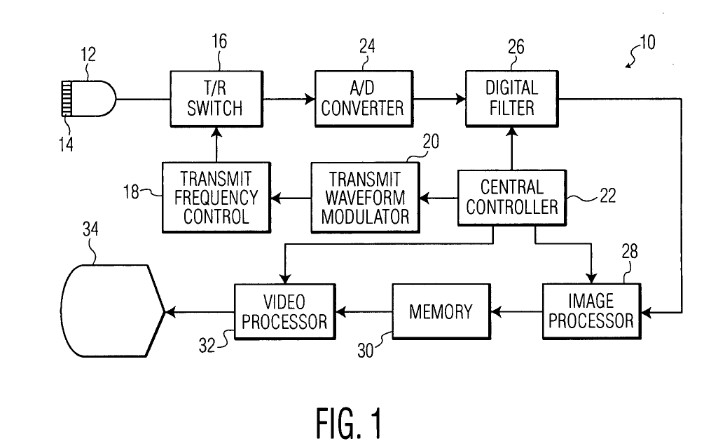

FIG. 1 is a block diagram of an ultrasound diagnostic imaging system constructed to implement the contrast imaging as taught herein;

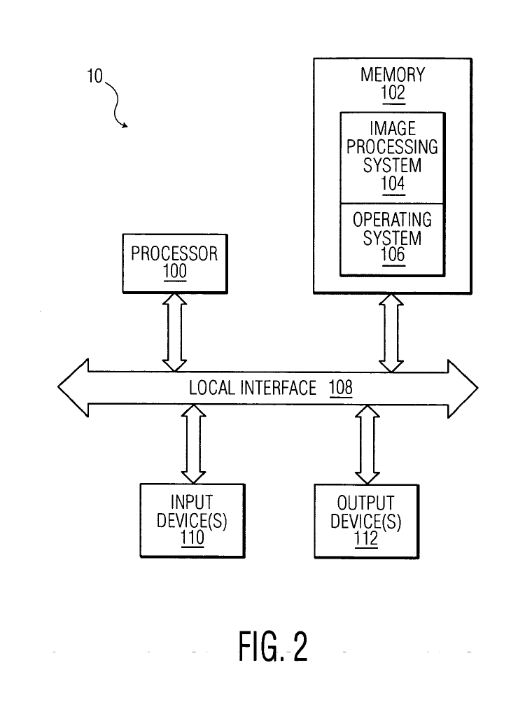

FIG. 2 is a functional block diagram of the system shown in FIG. 1;

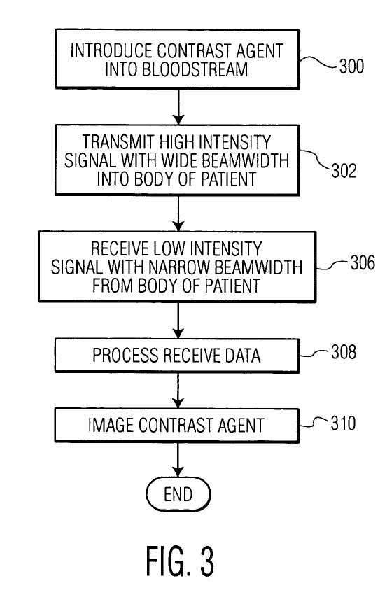

FIG. 3 is a flow diagram of a method for contrast imaging of the present invention;

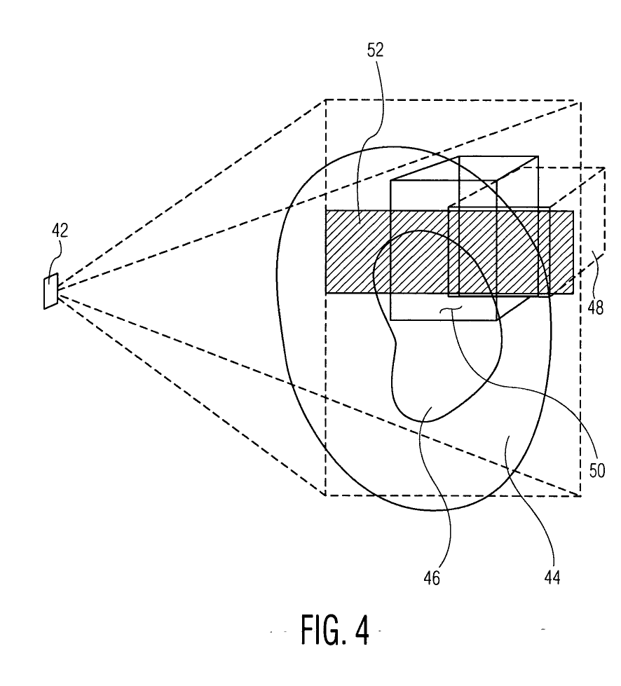

FIG. 4 is a schematic view of a placement of an ultrasound transducer relative a heart under investigation, with which the contrast imaging of the invention may be implemented;

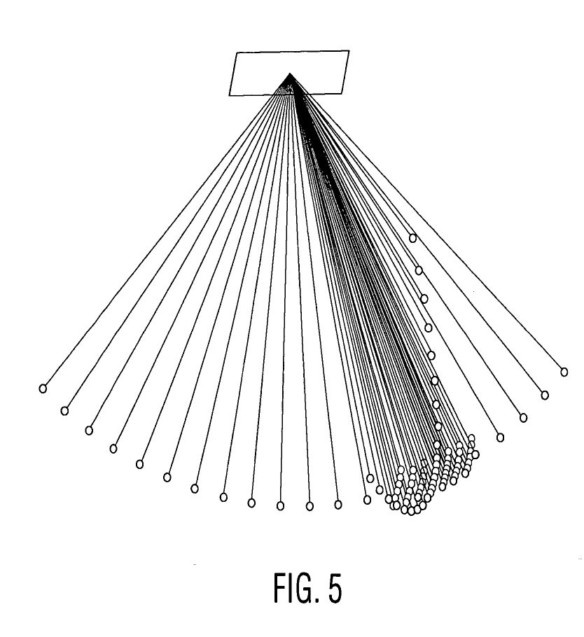

FIG. 5 is representation of a biplane scan with a reference volume centered about the intersection of two biplane images; and

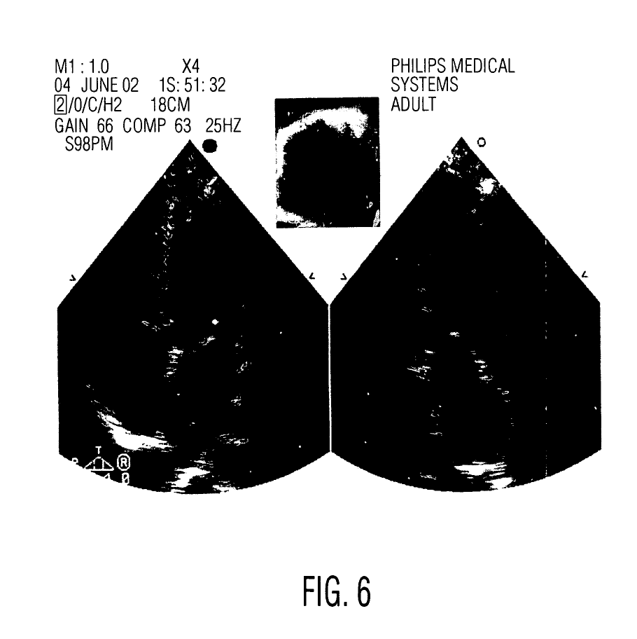

FIG. 6 is a display shot of a c-scan image display from a scan arrangement such as that set forth in FIG. 5.

These descriptions and representations are the means used by those skilled in the art to effectively convey the substance of their work to others skilled in the art. As used herein, a routine is generally conceived to be a self-consistent sequence of steps or actions leading to a desired result. Thus the term “routine” is generally used to refer to a series of operations performed by a processor, be it a central processing unit of an ultrasound system, and as such, encompasses such terms of art as “program,” “objects,” “functions,” “subroutines”, and “procedures.”

In general, the sequence of steps in the routines requires physical manipulation of physical quantities. Usually, though not necessarily, these quantities take the form of electrical or magnetic signals capable of being stored, transferred, combined, compared or otherwise manipulated. Those of ordinary skill in the art conveniently refer to these signals as “bits,” “values,” “elements,” “symbols,” “characters,” “images,” “terms,” “numbers” or the like. It should be recognized that these and similar terms are to be associated with the appropriate physical quantities and are merely convenient labels applied to these quantities. As used herein, the routines and operations are machine operations to be performed in conjunction with human operators. Useful machines for performing the operations of the present invention(s) include Philips' SONOS ultrasound systems, and other similar devices. In general, the present invention relates to method steps, software, and associated hardware including computer readable medium, configured to store and/or process electrical or other physical signals to generate other desired physical signals.

The apparatus as taught hereby is preferably constructed for the required purpose, i.e., an improvement in the arts of ultrasound cardiac perfusion imaging, using contrast agents, but the methods recited herein may operate on a general purpose computer or other network device selectively activated or reconfigured by a routine stored in the computer and interface with the necessary ultrasound imaging equipment. The procedures presented herein are not inherently related to any particular ultrasound system, computer or other apparatus. In particular, various machines may be used with routines in accordance with the teachings herein, or it may prove more convenient to construct more specialized apparatus to perform the required method steps. In certain circumstances, when it is desirable that a piece of hardware possesses certain characteristics, these characteristics are described more fully in the following text. The required structure for a variety of these machines may appear in the description below.

In one exemplary form, the present invention provides, inter alia, a process or routine (referred to hereinafter as process or method) for performing ultrasound perfusion imaging using ultrasound contrast agents in order to more accurately identify blood volumes while imaging with contrast agents. That is, the inventions improve the ability to “contrast image”. In system form, the inventions provide systems, inter alia, for contrast agent imaging a ROI with a high concentration of contrast agent where the flow into the ROI with contrast-agent-depleted blood is monitored and quantified, and movement of contrast-agent-laden blood present within tissue from outlying regions is prevented from moving into the ROI (or same movement is minimized) and inadvertently affecting the system's ability to accurately quantify perfusion in the ROI.

The inventions include imaging a “volume” or “region” within tissue for which perfusion analysis with contrast agent will be conducted, e.g., a region of interest (ROI) in the heart. To accurately detect and image perfusion data, volumes which supply blood to the ROI are also scanned to identify a particular blood supply volume, i.e., a destruction volume. Imaging the destruction volume with high MI ultrasound will destroy or render ineffective for contrast imaging the contrast agent in the laden-agent-laden blood present in the tissue and vasculature within the destruction volume or region. So the inventions identify a volume which supplies blood to the ROI, and destroys the contrast agent in blood therein as a “control” in detecting and quantifying perfusion in the ROI. The size and location of both the destruction zones and ROI is user controllable (the user also controls the MI of the scanning via the Ul), and is preferably and most effectively carried out by way of 3D imaging. Contemporaneously at the commencement of the high MI scanning of the destruction volume, imaging of the ROI with low MI ultrasound begins in order to image as the contrast-agent-depleted blood perfuses the imaging volume or region of interest, and the contrast-bearing blood that was present in the ROI at the commencement of destruction imaging is washed out of the region by the flow of contrast-agent-depleted blood thereunto. And while the inventions may be implemented using 2D scanning, they are understood to be most effective when used with 3D based scanning hardware and techniques.

3D imaging is known to those skilled in the art to require first generating a volume of ultrasound data (a 3D scan data set), which must be created by either scanning or scanning with interpolation. The volume data is then processed to create an image for display on a 2D surface, the image having at least an appearance of 3D (rendered). For example, one known 3D imaging method includes performing multiple sweeps wherein each sweep is oriented to a different scan plane. The planes of successive sweeps may be displaced with respect to each other in the elevation direction. Successive scans may be undertaken across the lateral axis, or about a centerline of the lateral dimension. In general, though, each scan frame comprises a plurality of lines allowing interrogation of a 3D scan data set representing a scan volume of some predetermined shape, such as a cube, cone, frustrum, cylinder, etc.

The detailed description which follows describes in detail how the inventors have improved on the art of ultrasound contrast perfusion imaging and analysis to realize the present inventions. The text and drawings present the invention(s) in terms of routines and symbolic representations of operations of data bits within a memory, associated processors, and possible networks, and network devices.

Referring now in more detail to the drawings, in which like numerals indicate corresponding parts throughout the several views, FIG. 1 illustrates an ultrasound contrast imaging system 10 by which the ultrasound contrast agent imaging contemplated by the invention is implemented. It will be appreciated that this figure does not necessarily illustrate every component of the system, emphasis instead being placed upon the components most relevant to the contrast imaging methods disclosed herein. The FIG. 1 system 10 includes a probe 12 comprising a transducer array of matrix elements 14 (a two-dimensional matrix array), used to transmit and receive ultrasound signals. The probe 12 is electrically connected to a T/R switch 16, which places the probe in a transmit mode or a receive mode. On the transmit side, the system 10 includes a transmit frequency control 18, and a transmit waveform modulator 20, that, under the control of a central controller 22, sets the transmit frequency of the transmit signals and modulates the various transmitted signal lines, respectively.

On the receive side, the system 10 includes an A/D converter 24 which converts the analog signals received from the probe 12 (the electrical signals generated by the transducer elements in receive mode) into digital signals. A digital filter 26 (e.g., an RF filter) filters signals outside the desired receive band from the received data. In addition, the receive side includes an image processor 28, which can record the data for tissue motion effects and can, for instance, suppress the stationary tissue signal components (in a harmonic imaging mode). The corrected data can be stored in a memory 30 and, after being processed by a video processor 32, displayed on a display device 34.

FIG. 2 illustrates several pertinent functional components of the ultrasound contrast imaging system 10, shown in FIG. 1. As depicted in FIG. 2, the system 10 typically comprises a processor 100, a memory 102, a local interface 108, and an output device 112. The memory 102 includes, inter alia, an image processing system 104, as well as an operating system 106. Furthermore, the memory 102 can include the memory 32 shown in FIG. 1. As will be appreciated by those having ordinary skill in the art, the image processing system 104 can be implemented in software, hardware, or a combination thereof, within the image correction processor 30 shown in FIG. 1. It is to be noted that when implemented in software, the system 104 can be stored and transported on any computer readable medium for use by or in connection with an instruction execution system, apparatus, or device, such as a computer-based system, processor containing system, or other system that can fetch the instructions from the instruction execution system, apparatus, or device and execute the instructions.

In the context of this disclosure, a “computer readable medium” can be any means that can contain, store, communicate, propagate, or transport the program for use by or in connection with the instruction execution system, apparatus, or device. The computer readable medium can be, for example, an electronic, magnetic, optical, electromagnetic, infrared, or semiconductor system, apparatus, device, or propagation medium. More specific examples of computer readable media include the following: an electrical connection having one or more wires, computer diskette, random access memory (RAM), read only memory (ROM), erasable programmable read only memory (EPROM or Flash memory), an optical fiber, and a compact disk read only memory (CD ROM). It is to be noted that the computer readable medium can even be paper or another suitable medium upon which the program is printed as the program can be electronically captured, via for instance optical scanning of the paper or other medium, then compiled, interpreted, or otherwise processed in a suitable manner if necessary, and then stored in a computer memory.

The reader is now referred to FIG. 3, which depicts an exemplary embodiment of a contrast imaging method of the present invention, and to FIG. 4, which depicts an imaging example, using the process set forth in the method. As indicated in block 300, a contrast agent is first introduced intravenously into the patient's bloodstream.

The contrast agent may comprise micro-bubbles of a heavy gas, such as a perfluorocarbon gas encapsulated in an outer shell made of protein, lipid, or other suitable material. Although the size of the agent may vary depending upon the application, these micro-bubbles normally are in the range of approximately 1.0 to 15 microns (μm) in diameter. As the contrast agent is introduced into the bloodstream, it travels through the right side of the heart and passes through the capillaries in the lungs. The contrast agent (micro-bubbles) normally moves through the blood pathways to a tissue ROI, for example, a human heart.

Flow of contrast-agent-laden pulmonary blood through the left ventricle (LV), part of the pumping cycle, also supplies the coronary circulation system. The contrast agent, i.e., micro-bubbles, is delivered to the myocardium by the circulation. Imaging with contrast agents (contrast imaging), as mentioned above, has limitations in certain circumstances, for example, for imaging the myocardium behind or proximate the LV. Any limitation or inaccuracy in data derived by the contrast imaging consequently limits the value of the data in making a useful diagnosis. An ability to detect and quantify blood flow, by presence or absence of contrast-agent-laden blood, or by volume changes over time, provides the ability detect ischemic regions in the myocardium.

The circulation of blood in the myocardium is fairly slow, such that if the contrast agent is destroyed during one or more destruction frames, replenishment of the myocardial region is typically for a normally functioning myocardial region on the order of four or five heart cycles, or about four or five seconds for an adult human. For an ischemic region where blood flow is reduced, replenishment can take significantly longer than in normal healthy tissue. By viewing the intensity of the contrast agent in the image plane overtime, a replenishment curve can be generated, which essentially is an exponential increasing curve in which a region is related to the rate of blood flow in the myocardium. The rate of blood flow is a parameter of significant clinical interest because under certain conditions, it can indicate viability of the myocardium or the amount of ischemic damage to the region, which is being imaged.

Step 300 further includes imaging and defining an ROI, for example, in the myocardium behind or proximate the left ventricle, using low MI frames. A determination is also undertaken to identify a volume proximate the ROI through or by which blood perfuses the ROI, which is adjustable. For example, by first identifying a destruction plane through which a major conduit to/from the LV flows through relatively perpendicularly to the plane or volume.

Then, as indicated in block 302 of FIG. 3, after the contrast-agent-laden blood has reached the ROI, for example, and surrounding myocardium (as shown in FIG. 4), high MI ultrasound signals are transmitted into the destruction volume to destroy contrast agent within the contrast-agent-laden blood therein.

FIG. 4 illustrates the positioning of a transducer 42, with respect to a heart 44, the perfusion of which is under investigation. For example, if myocardium within a ROI, 48, located behind left ventricle 46, is to be perfusion imaged, blood containing contrast agent flows into the left ventricle and then subsequently (the next pump cycle) into the arterial pathways of the myocardium (and therefore into the ROI). A destruction zone or region 50 is defined for destruction imaging so that contrast agent within blood present in the destruction volume at which the high MI scanning from the transducer destroys the ultrasound contrast agent. The destructive signals must deliver signals at a sufficiently high acoustic pressure (which is in the range of current ultrasound systems) to disrupt the shell of the micro-bubbles and cause the gas which is encapsulated to be dissolved into the bloodstream, and surrounding tissue relative imaging plane 50. The time for the dissolving of the gas into the blood stream differs based on the composition of the gas, but essentially the gas will re-absorb into the blood stream quite rapidly relative to the time involved for the blood to travel from the destruction region to the ROI, and may be within the time to acquire a single frame of a real time ultrasound exam.

As indicated in block 306, the low MI ultrasound from an ultrasound transducer 42 of ultrasound imaging system 10 of FIG. 1 scans the ROI 48 substantially simultaneously with the high MI scanning of the destruction zone 50. The low MI scanning of the ROI 48 may be conducted during the high MI scanning, or immediately thereafter. Perfusion imaging is accomplished in the tissue ROI, e.g., behind or proximate the LV 46, where there is substantially no contrast-agent-laden blood perfusing the ROI from the destruction zone. When implemented as taught hereby, the result is an imaging method which minimizes error due to contrast agent in blood present in a zone feeding the ROI, or merely present in the tissue surrounding the ROI (due to mechanical movement of tissue surrounding the ROI) from moving into the ROI 48, as 3D volume imaging is conducted by the ultrasound system. That is, imaging the ROI 48 with low MI ultrasound as contrast agent-depleted blood perfuses the ROI 48 from the destruction zone 50 provides data relating to blood volumes immediately, and over time, without the deleterious effects inherent in prior art aforementioned.

The destruction zone or volume 50 may be thicker or thinner in the elevation direction (as shown in FIG. 4) than an image volume 52. The only limitation in this respect is as understood by those skilled in the art. Both the image volume 52 and the destruction zone 50 may be pie shaped sectors of a plane through the area of interest, for example, the heart. These planes have a thickness determined by the elevation focusing of the ultrasound system 10, and vary with distance from the ultrasound transducer 42. By broadening the elevation focus in the elevation direction during the destruction frames by using, for instance, fewer sub-elements of the ultrasound transducer 42, the contrast agent is destroyed in a wide area and substantially prevents the contrast agent from reentering the image volume 52 or ROI 48 from motion during subsequent image frames. Any contrast agent that lies outside of the destruction volume is not destroyed. The destruction volume 50, which represents the wide area in which the contrast agent is destroyed during the destruction frames, may or may not be larger than the imaging volume 52 in which contrast agent is imaged during the imaging frames.

By destroying a thicker slice of the contrast-agent-laden tissue (blood therein) surrounding the image volume or ROI 48, mischaracterizations of blood volume or perfusion of the ROI by contrast-agent-laden blood in the tissue surrounding the ROI inadvertently moving in due to motion of the patient (or other internal movements) is minimized (not mistaken for reperfused blood therein). The result is a more accurate measure of the rate of reperfused blood flow to or through the ROI. The destruction frames are typically only used for destroying the contrast agent and not for imaging, but this is not necessarily always the case. Please note, however, that the aforementioned dimensions are for exemplary purposes only, and not to limit the scope of applicants' inventions in any way.

As indicated in block 308 (FIG. 3), the received image echoed from the patient is imaged using contrast data processing techniques. As indicated in block 310, the images can be provided to a video display, or other output device for viewing. So, where the narrower image plane is imaged either immediately or a very short period of time after a destruction frame, then the image will be of tissue in the ROI enhanced by contrast agent, and into which the flow of blood which does not contain contrast agent may be observed. Depending on obstructions, there may or may not be an observed decrease in contrast agent in the ROI with time.

As mentioned, transducer 42 may be controlled to first image the heart 44 along a long axis parasternal plane in a normal investigational manner. An imaging volume or ROI, and a destruction volume may be chosen through user input, or automatically, such that a portion of the imaging volume is made to intersect with a majority of blood flow through the left ventricle 46. Specifically, the blood flow enters the left ventricle by the mitral valve and exits through the aortic valve. To do so, the user must define the imaging plane or region using some kind of segmentation procedure available on the particular system being utilized. For example, the user should define a region of plane (e.g., defining a plane between the mitral and aortic valves) by use of some kind of pointing device (not shown in FIG. 4) or other segmentation method or device known to those skilled in that art. In the present example, the user would outline the destruction region 50, which is to be scanned by the high power ultrasound signals.

The segmentation merely provides that pixel coordinates defining the destruction volume 50 or ROI 48 to the signal processing means for later use to manage control and timing. Thereafter, when it is determined that scanning beams emitted from transducer 46 enters the destruction region 50 based on the stored coordinate values, a power change signal is dispatched to transmit control means to direct higher power ultrasound signals to the transducer 42. The higher power signals are scanned across the destruction region 50 and destroy the contrast agent in the region only. The remaining regions of the myocardium are subsequently, but almost contemporaneously imaged using the low-MI contrast detection technique. After commencement of the high-MI frames in the blood path, the concentration of contrast agent in the blood flowing is significantly reduced (ideally to 0) due to the destruction by the acoustic pressures in the destruction region 50.

The lack of contrast agent within blood flowing into the ROI 48 causes a significant change, e.g., reduction, in the concentration of contrast agent detected in the ROI by low MI. These changes provide a good indication of the characteristics of the perfusion of the cardiac regions of interest. Accordingly, the signal intensity from contrast agent in the myocardium will drop exponentially. That is, by measuring the negative change in intensity over time, a perfusion curve may be gleaned and quantified. The perfusion curve showing the decreases of contrast agent concentration as non-contrast agent-enhanced blood flows into the ROI, displacing contrast agent-laden blood may be readily calculated.

Such a method is more advantageous than the conventional negative step function method for at least two reasons. First, the concentration of contrast agent in the ventricle is considerably reduced by the high power scanning. The apparent result is that ultrasound beams passing through the ventricle intended for low-MI detection of contrast agent experience less distortion as they pass through the ventricle. Second, all regions of the myocardium should theoretically have the same concentration of contrast agent, which decreases only as a function of the concentration of contrast agent being fed by the coronary arteries, and the rate of blood flow in the regions under investigation. Accordingly, there will be significantly less error in perfusion estimates caused by motion of surrounding tissue regions into the imaging plane.

As mentioned above, a preferred embodiment utilizes 3D scanning techniques such that a small configurable volume scan could be positioned over either the mitral or aortic valves by the user. This 3D imaged volumetric region would then be used with the high-MI to control the concentration of contrast agent out of the ventricle such that the acoustic power in the sub volume may be controlled independently by the user from other regions via the UI. Simultaneously with the small volume region scanned, one or more planes or regions can be scanned in outlying areas, using the low-MI contrast detection techniques. There is no limitation intended, however, for the scanning can be implemented as a single scan plane, multiple planes, or volume scans. The multiple planes and volumes can be user controlled to view different coronary regions of interest. For that matter, a separate transducer may be used for high MI, and for low MI imaging as taught herein.

In another embodiment, the present invention includes an imaging method which scans a biplane plus a small, movable reference volume and uses the reference volume's scan lines to create a C-scan that is automatically aligned with the biplane. The C-scan may be generated using 3D rendering techniques known to those skilled in the arts, or it may be implemented by a simple inventive technique which defines the C-scan without the need for 3D rendering. That is, the invention teaches that reference volume lines are generated differently than the bi-plane lines and therefore used in color flow imaging and contrast imaging.

In particular, the present embodiment envisions scanning the bi-plane and reference volume together; such that only the scan lines needed for developing the three images (2 planes and C-scan) are actually scanned to facilitate a contrast perfusion exam. By scanning in this manner, instead of scanning the whole encompassing volume, the frame rate may be maximized. For that matter, the invention requires that the bi-plane image pair and the reference volume automatically track a cursor dot so that the user may easily select and examine tissue structures from three perspectives (the UI). The invention allows the user to identify one or both of the bi-planes as an imaging plane, and define one or both planes such that a portion or region of one or both planes are arranged to intersect with a majority of blood flow into the volume (which may or may not include the left ventricle). For example, one of the planes could be located at the mitral or aortic valves, so that high energy ultrasound focused on one or both of the planes such that, for example, blood flowing out of the heart through aortic valve into the C-scan volume will have its content of contrast agent reduced to near zero.

This causes a negative step function in the concentration of contrast agent feeding the coronary circulation (i.e., the volume of the C-scan) and ultimately the perfusion in the volume. As such, the signal intensity from contrast agent in the myocardium will drop in an exponential fashion, so measuring the intensity over time, a perfusion curve for the volume defined by the biplanes may be generated. More particularly, the user interface allows the user to control the biplane and the depth of the C-scan formed thereby using 3D scanning techniques (as known in the art), such that the volume is positioned over either the aortic or mitral vale to control contrast agent in the left ventricle. That is, the volume is scanned with high energy to destroy the contrast agent.

The reader should note, however, that the inventions herein are not limited to the embodiments described. The present invention may further include that while the small volume is scanned, one or more planes in outlying areas (e.g., a single scan plane, multiple planes, or volume scans), which may be controlled by the user to view various coronary territories of interest are scanned with low MI ultrasound.

FIG. 5 shows an embodiment of the invention, which includes both a biplane and a reference volume. The combination derives from a biplane scan including two image planes orthogonal to each other and the plane of the array. FIG. 6 shows a biplane plus C-scan image display, where the small diamond, seen in the image above the mitral valve in the left biplane indicates the tilt of the right biplane as well as the depth of the C-scan. From the reference volume scan, the system extracts a C-scan and displays the volume simultaneously with the two biplane images. Extraction consists of simple windowing of acoustic samples from the scan lines of the reference volume, with optional filtering in depth to create the selectable C-scan slab thickness in the display. No sophisticated 3D rendering hardware or software is needed, i.e., no ray casting or shear warp processing.

It is preferable that the system user interface show a cursor dot or diamond on the left plane that both controls and indicates the tilt of the right plane, but with a new addition, it must also indicate/control the depth of the c-scan displayed alongside the bi-plane. Accordingly, a user may place the cursor on a structure, such as the aortic valve, and the system will adjust itself to view the structure from three perspectives simultaneously. The user may then utilize the views to identify the volume for destruction of contrast agent using high-MI frames.

FIGS. 5 and 6 further highlight that the reference volume is centered about the line of intersection of the two planes, such that is should be apparent that the user interface preferably includes a control for lateral and/or elevation translation of the reference volume with respect to the feline of intersection. This may be characterized as a C-scan pan function, useful because the C-scan is small enough that the user might want to move the downward-looking view around like a magnifying window, studying the outlying areas of the structure while holding the biplane as a locational reference. For that matter, the scan lines of the reference volume may be specialized, e.g., their transmit/receive parameters may be selected for visualizing contrast agent, or releasing active pharmaceuticals comprising the contrast.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

1. A method of perfusion analysis using ultrasound contrast agents, comprising the steps of:
introducing contrast agent into the bloodstream of a patient under examination in order to perfuse a volume of interest;
identifying the volume of interest and a destruction volume, proximate the volume of interest, which supplies blood to the volume of interest, by scanning the volume of interest and destruction volume using low mechanical index (MI);
destroying at least the contrast agent in the blood supply within the destruction volume using high MI ultrasound in order to destroy contrast agent within blood present in the destruction volume such that said present blood is contrast-agent-depleted, which destruction volume includes the blood supply; and
imaging the volume of interest using low MI ultrasound substantially simultaneously as the contrast-agent-depleted blood from the destruction volume perfuses the volume of interest, causing an exchange within the volume of interest, of contrast-agent-laden blood with contrast-agent-depleted blood.

2. The method set forth in claim 1, wherein the step of imaging includes quantifying blood volumes present in the volume of interest.

3. The method set forth in claim 1, wherein the step of imaging includes quantifying velocity of blood flowing within the volume of interest.

4. The method as set forth in claim 1, wherein the step of identifying further includes identifying the destruction volume to include tissue, proximate the volume of interest, from which destruction volume contrast-agent-laden blood located in the tissue could move into the volume of interest during the step of imaging, and destroying same in said destruction volume to prevent the blood from seeping into the volume of interest.

5. The method as set forth in claim 1, where blood flow within the volume of interest is a cardiac volume.

6. The method as set forth in claim 1, wherein the step of imaging includes identifying restricted blood flow within the volume of interest.

7. The method as set forth in claim 1, wherein the step of identifying includes defining volumetric parameters which define the volume of interest using a biplane scan process.

8. The method as set forth in claim 7, wherein the volumetric parameters include the location of each biplane.

9. The method as set forth in claim 8, wherein the volume of interest is bounded by each plane of the biplane.

10. The method as set forth in claim 1, wherein the step of identifying is user adjustable.

11. The method as set forth in claim 3, wherein said velocity information is displayed.

12. The method as set forth in claim 11, wherein said velocity information is displayed as a color image, and velocity information is color-coded.

13. The method of claim 1, where the destruction region is defined by a c-scan plane, and the ROI is contained within the biplane.

14. A computer readable medium within which is encoded a set of computer instructions, which set provides for the implementation of the method as set forth in claim 1.

15. A method for conducting ultrasound perfusion studies on myocardial tissues utilizing ultrasound contrast agents, comprising the steps of:
infusing a patient's blood with contrast agent;
transmitting low-MI ultrasound pulses into a region of interest (ROI) within the patient's heart, and receiving ultrasound echoes of the pulses, to highlight the presence of blood infused with the contrast agent in myocardial tissues disposed in the ROI;
determining an imaging volume within the ROI, from which it is desired to quantify perfusion data for the imaging volume;
determining a destruction volume within the ROI, proximate the imaging volume, which destruction volume is determined to include substantially any blood, which might perfuse the imaging volume in the region;
transmitting high-MI ultrasound pulses to the destruction volume to destroy all contrast agent present therein, and substantially simultaneously imaging the imaging volume using low-MI ultrasound pulses; and
calculating perfusion data for the imaging volume based on an assessment of a rate of decrease of a volume of contrast-agent-laden blood therein as the contrast-agent-depleted blood perfuses the imaging volume.

16. The method as set forth in claim 15, wherein the step of calculating is assumed to be accurate in accordance with a detection of a reperfusion, with contrast-agent-laden blood, of the destruction volume subsequent to cessation of the imaging therein with high MI ultrasound.

17. The method as set forth in claim 15, wherein the step of determining further includes identifying a destruction volume through which contrast-agent-laden blood flows into the imaging volume.

18. The method as set forth in claim 17, further including imaging said destruction volume to prevent contrast-agent-laden blood from moving into the imaging volume.

19. The method as set forth in claim 15, wherein said region is identified using a biplane scan process which produces a small c-scan relative the location of said biplanes.

20. The method as set forth in claim 17, wherein said destruction volume is defined by one of said biplanes.

21. The method as set forth in claim 19, wherein said C-scan includes the imaging volume.

22. A ultrasound imaging system for conducting perfusion analysis of myocardial tissue volumes utilizing ultrasound contrast agents, comprising:
an ultrasound transducer for transmitting low-MI ultrasound pulses into a region of a patient's heart, and receiving ultrasound echoes of the pulses, to highlight the presence of blood infused with the contrast agent in the region; and
a selector within a user interface to the ultrasound system for allowing a user:
1) to choose an imaging volume in the region,
2) to choose a destruction volume within the region, wherein the destruction volume is a volume in th region proximate the imaging volume through which contrast-agent-laden blood perfuses the imaging volume,
3) to automatically scan the destruction volume with high MI ultrasound to destroy contrast agent in blood present therein, and
4) to automatically scan the imaging volume with low MI ultrasound;
wherein said automatic scan of said imaging volume is conducted substantially immediately at completion of said automatic scanning of the destruction volume to detect a perfusion of non-contrast-agent-laden blood thereinto.

23. The imaging system set forth in claim 22, wherein the selector allows the user to implement a biplane with small c-scan volume mode of imaging, by which the user selects whether the c-scan is the destruction volume or the imaging volume.

24. The imaging system set forth in claim 22, wherein the selector allows the user to select a size and location of one of the imaging volume and destruction volume.

25. The imaging system set forth in claim 22, wherein the selector allows the user to select the MI implemented in any transmission of ultrasound.

26. A ultrasound imaging system for conducting perfusion analysis of myocardial tissue volumes utilizing ultrasound contrast agents, comprising:
an ultrasound transducer for transmitting low-MI ultrasound pulses into a region of a patient's heart, and receiving ultrasound echoes of the pulses, to highlight the presence of blood infused with the contrast agent in the region; and
a selector within a user interface to the ultrasound system for allowing a user:
5) to choose an imaging volume in the region,
6) to choose a destruction volume within the region, wherein the destruction volume is a volume in th region proximate the imaging volume through which contrast-agent-laden blood perfuses the imaging volume,
7) to automatically scan the destruction volume with high MI ultrasound to destroy contrast agent in blood present therein, and
8) to automatically scan the imaging volume with low MI ultrasound;
wherein said automatic scan of said imaging volume is conducted substantially immediately at completion of said automatic scanning of the destruction volume and includes scanning at least two intersecting planes and a related volume to detect a perfusion of non-contrast-agent-laden blood thereinto.

27. The ultrasound imaging system of claim 26, wherein the related volume is a reference volume whose center tracks the intersection of one of the at least 2 intersecting planes.

28. The ultrasound imaging system of claim 27, wherein the system extracts a C-scan developed from the reference volume and displays it simultaneously with multi-plane images corresponding to the at least 2 intersecting planes.

29. The ultrasound imaging system of claim 26, further including a user interface (UI) which provides one of a cursor dot, diamond or like indicator on at least one displayed plane of the two intersecting planes, which indicator at least one of: controls the tilt of the another plane, and indicates the tilt of another plane, and further indicates/controls the depth of the C-scan displayed alongside the multi-plane images.

30. The ultrasound imaging system of claim 28, further including an I-scan instead of a C-scan, with user control over the incline angle and direction.

31. The ultrasound imaging system of claim 27, wherein the center of the reference volume is offset from the line of intersection by an amount and in a direction controlled by the user interface.

32. The ultrasound imaging system of claim 28, wherein a spatial extent and a scan line density are configured by the user, trading off image quality, frame rate, and viewing extent.

33. The ultrasound imaging system of claim 28, wherein the reference volume acquires Color Flow scan lines and/or black and white, and the C-scan displays Flow.

34. The ultrasound imaging system of claim 33, wherein the reference volume has scan line transmit/receive parameters selected for visualizing contrast agent and/or releasing pharmaceuticals.

35. The ultrasound imaging system of claim 26, wherein the automatic scanning generates a c-scan using AQ, to determine a real-time valve volume, with on-screen border indication and volume measurement output.

36. The ultrasound imaging system as set forth in claim 35, wherein real-time-color flow may be implemented over the real-time valve volume.

37. The ultrasound imaging system of claim 35, wherein the user may be provided with real-time image information defining a flow value, which flow value represents the integral of the flow from the real-time valve volume.

38. The ultrasound imaging system of claim 37, wherein said real-time image information includes a graph of the flow value over time.

39. The ultrasound imaging system of claim 38, further including that the user interface include a mechanism for varying a look angle relative the valve area to maximize flow detection.

40. The method set forth in claim 1, wherein said steps of destroying and imaging are implemented on one of a frame interleave and a line interleave basis.

41. The method set forth in claim 15, wherein said step of transmitting includes frame interleaving said high and low MI frames.

42. The imaging system as set forth in claim 22, wherein said selector automatically scans the destruction and imaging volumes on a frame interleaved basis.

43. The imaging system as set forth in claim 22, wherein the transducer is a TEE probe comprising a 2D array.

44. The imaging system as set forth in claim 43, wherein the transducer is a TEE probe comprising a matrix array.
